(19) United States
(12) Patent Application Publication
(10) Pub. No.: US 2024/0075310 A1
(43) Pub. Date: Mar. 7, 2024

(54) Targeting Trancranial Magnetic Stimulation to Specific Brain Regions and Evaluating the Reduction of Symptoms of Psychotic Disorders

(71) Applicant: Beth Israel Deaconess Medical Center, Inc., Boston, MA (US)

(72) Inventors: Roscoe Brady, Jr., Cambridge, MA (US); Mark Halko, Natick, MA (US)

(21) Appl. No.: 18/256,739

(22) PCT Filed: Dec. 9, 2021
(86) PCT No.: PCT/US2021/062670

§ 371 Date: Jun. 9, 2023

Related U.S. Application Data
(60) Provisional application No. 63/123,843, filed on Dec. 10, 2020.

(51) Int. Cl. A61N 2/00; A61N 2/02; G01R 33/48

(52) U.S. Cl. CPC A61N 2/006; A61N 2/02; G01R 33/4806

(57) ABSTRACT

The present disclosure describes a computerized system for a method that includes: accessing brain images of a patient, e.g., a patient who has been diagnosed with a psychotic disorder, or at high risk for developing the psychotic disorder, wherein the brain images comprise a first set of images depicting a brain structure of the patient and a second set of images encoding a brain function of the patient; generating masks based on morphing a symptom region of an atlas to the first set of images, wherein the atlas is based on a cohort of participants with the psychotic disorder; identifying treatment regions based on applying the one or more masks to the second set of images, wherein the treatment regions are specific to the patient and the symptom; and causing at least one transcranial magnetic stimulation (TMS) therapy session targeting the one or more treatment regions to be administered.

### CLAIM OF PRIORITY

This application claims the benefit of U.S. Provisional Patent Application Ser. No. 63/123,843, filed on Dec. 10, 2020. The entire contents of the foregoing are hereby incorporated by reference.

### STATEMENT OF GOVERNMENT FUNDING

This invention was made with government support under grant nos. MH092440 and MH111868 awarded by the National Institutes of Health. The government has certain rights in the invention.

### TECHNICAL FIELD

This disclosure generally relates to the management of psychotic disorders, such as schizophrenia.

### BACKGROUND

Schizophrenia is chronic psychotic disorder associated with tremendous mortality and disability. Schizophrenia is a highly heterogeneous condition and individuals with this disorder vary considerably in their symptoms. These symptoms can be grouped into four different categories, namely, psychotic symptoms (e.g. hallucinations), negative symptoms (asociality, amotivation), cognitive deficits, and mood symptoms. Existing medication treatments for schizophrenia, known as anti-psychotics, only treat the psychotic symptoms without efficacy in treating ‘negative’ symptoms or cognitive deficits which are the most disabling symptoms of schizophrenia. Further, these medications are hampered by poor efficacy and tolerability.

### SUMMARY

In one aspect, the present disclosure describes a computer-implemented method comprising: accessing brain images of a patient who has been diagnosed with a psychotic disorder or at high risk for developing the psychotic disorder, wherein the brain images comprise a first set of images depicting a brain structure of the patient and a second set of images encoding a brain function of the patient; generating one or more masks based on morphing a symptom region of an atlas to the first set of images, wherein the symptom region of the atlas is identified from a cohort of participants, e.g., subjects with a psychotic disorder; identifying one or more treatment regions based on applying the one or more masks to the second set of images, wherein the treatment regions are specific to the patient and a symptom of the psychotic disorder; and prescribing or causing at least one transcranial magnetic stimulation (TMS) therapy session targeting the one or more treatment regions to be administered to a brain of the patient. In some embodiments, the patient is a patient who is suspected of having a psychotic disorder, who has been diagnosed with a psychotic disorder, or who is at high risk for developing the psychotic disorder. In some embodiments, the patient has a diagnosed psychotic disorder with symptoms such as hallucinations or negative symptoms of schizophrenia. In other embodiments, the patient is in a prodromal stage of a psychotic disorder, and has symptoms such as cognitive impairment e.g. reduced information processing speed.

Implementations may include one or more of the following features.

The method may further include: accessing a third set of images of the patient that encode the brain function of the patient after the at least one TMS therapy session is administered. The method may further include: applying the one or more treatment regions to the third set of images; and evaluating an effect of the at least one TMS therapy session based on results of correlating a signal from the one or more treatment regions and a signal from at least one node that is downstream of the one or more treatment regions. The method may further include: causing at least one more TMS therapy session targeting the one or more treatment regions to be administered to the patient, wherein at least one stimulation parameter of the at least one more TMS therapy session is updated based on results of the evaluation. The first set of images may be characterized by a spatial resolution higher than the second set of images. The second set of images may include functional magnetic resonance imaging (fMRI) images that encode the brain function of the patient.

The method may include: removing signals of non-neuronal origin from the fMRI images based on preprocessing. Identifying one or more treatment regions may include: performing a correlation between an average blood oxygen level dependent (BOLD) signal from voxels within the one or more masks applied to the second set of images and a BOLD signal elsewhere in the second set of images. Identifying one or more treatment regions may further include: applying a threshold to results of the correlation such that the one or more treatment regions specific to the patient and a symptom of the psychotic disorder can be identified. The third set of images may include fMRI images encoding the brain function of the patient, wherein the third set of images and the second set of images are acquired using an identical protocol. The method may include: performing a correlation between an average BOLD signal from voxels within the one or more masks applied to the third set of images and a BOLD signal from voxels within a downstream node within the third set of images; and evaluating an effect of the at least one TMS therapy session based on results of the correlation.

The method may further include: guiding the at least one TMS therapy session based on the one or more treatment regions. The method may further include: generating a head-mounted template which, when mounted on the patient along with one or more TMS coil, causes the one or more TMS coil to focus on the one or more treatment regions within the brain of the patient.

In another aspect, the present disclosure describes a computer system including one or more processors configured to perform operations of: accessing brain images of a patient, e.g., a patient who has been diagnosed with a psychotic disorder or is at high risk for developing the psychotic disorder, wherein the brain images comprise a first set of images depicting a brain structure of the patient and a second set of images encoding a brain function of the patient; generating one or more masks based on morphing a symptom region of an atlas to the first set of images, wherein the symptom region of the atlas is identified from a cohort of participants with a psychotic disorder; identifying one or more treatment regions based on applying the one or more masks to the second set of images, wherein the treatment regions are specific to the patient and a symptom of the psychotic disorder; and prescribing or causing at least one transcranial magnetic stimulation (TMS) therapy session targeting the one or more treatment regions to be administered to a brain of the patient.

The operations may further include: accessing a third set of images of the patient that encode the brain function of the patient after the at least one TMS therapy session is administered. The operations may further include: applying the one or more treatment regions to the third set of images; and evaluating an effect of the at least one TMS therapy session based on results of correlating a signal from the one or more treatment regions and a signal from at least one node that is downstream of the one or more treatment regions. The operations may further include: causing at least one more TMS therapy session targeting the one or more treatment regions to be administered to the patient, wherein at least one stimulation parameter of the at least one more TMS therapy session is updated based on results of the evaluation.

The first set of images may be characterized by a spatial resolution higher than the second set of images. The second set of images may include functional magnetic resonance imaging (fMRI) images that encode the brain function of the patient. The operations may further include: removing signals of non-neuronal origin from the fMRI images based on pre-processing.

The operations may further include: performing a correlation between an average blood oxygen level dependent (BOLD) signal from voxels within the one or more masks applied to the second set of images and a BOLD signal elsewhere in the second set of images. The operations may further include: applying a threshold to results of the correlation such that the one or more treatment regions specific to the patient and the symptom can be identified. The third set of images may include fMRI images encoding the brain function of the patient, wherein the third set of images and the second set of images are acquired using an identical protocol. The operations may further include: performing a correlation between an average BOLD signal from voxels within the one or more masks applied to the third set of images and a BOLD signal from voxels within a downstream node within the third set of images; and evaluating an effect of the at least one TMS therapy session based on results of the correlation.

The operations may further include: guiding the at least one TMS therapy session based on the one or more treatment regions. The operations may further include: generating a head-mounted template which, when mounted on the patient along with one or more TMS coil, causes the one or more TMS coil to focus on the one or more treatment regions within the brain of the patient.

Implementations according to the present disclosure may be realized in computer implemented methods, hardware computing systems, and tangible computer readable media. For example, a system of one or more computers can be configured to perform particular actions by virtue of having software, firmware, hardware, or a combination of them installed on the system that in operation causes or cause the system to perform the actions. One or more computer programs can be configured to perform particular actions by virtue of including instructions that, when executed by data processing apparatus, cause the apparatus to perform the actions.

The details of one or more implementations of the subject matter of this specification are set forth in the description, the claims, and the accompanying drawings. Other features, aspects, and advantages of the subject matter will become apparent from the description, the claims, and the accompanying drawings.

### DESCRIPTION OF DRAWINGS

FIG. 1 illustrates an example of a diagram for obtaining an MM scan according to an implementation of the present disclosure.

FIG. 2 illustrates an example diagram for applying a mask to MM images from an individual to identify individually-determined and symptom-specific target regions according to an implementation of the present disclosure.

FIG. 3 illustrates an example of administering transcranial magnetic stimulation (TMS) to the individual based on the identified target regions according to an implementation of the present disclosure.

FIG. 4A illustrates an example of a diagram for analyzing follow-up functional MRI images to evaluate the effect of the TMS treatment according to an implementation of the present disclosure.

FIG. 4B illustrates preliminary results of changes in multiple symptoms and their associated circuits after rTMS therapy.

FIG. 5 illustrates an example of a flow chart according to an implementation of the present disclosure.

FIG. 6 is a block diagram illustrating an example of a computer system used to provide computational functionalities associated with described algorithms, methods, functions, processes, flows, and procedures, according to an implementation of the present disclosure.

Like reference numbers and designations in the various drawings indicate like elements.

### DETAILED DESCRIPTION

The disclosed technology is directed to a technique for planning and evaluating the treatment of psychotic disorders such as schizophrenia. Some implementations include planning treatment of such disorders by collecting and analyzing two types of images, namely, high-resolution structural image of the brain and low-resolution functional images of the brain. In some cases, the low-resolution functional images include an individual's functional magnetic resonance image (fMRI) data. In these cases, the two types of images are processed and analyzed to generate an individualized target, the location of which is specific to the individual patient's anatomy and the symptom targeted for amelioration. The processing can include isolating the blood oxygenation level dependent (BOLD) signal within a precisely defined three-dimensional region and performing a correlation between the average BOLD signal in this region and the BOLD signal in every other voxel in the individual's brain scan at every time point of the fMRI scan. The result of the correlation can provide the identification of an individually determined and symptom specific map with sufficient precision to define targets for transcranial magnetic stimulation (TMS). This individually determined and symptom specific map can serve as a target to guide TMS in multiple forms.

For context, transcranial magnetic stimulation (TMS) is a noninvasive procedure that uses magnetic fields to stimulate nerve cells in the brain to improve symptoms of depression, TMS is typically used when other depression treatments haven't been effective. To the extent that this treatment for depression involves delivering repetitive magnetic pulses, the treatment may also be known as repetitive THIS or rTMS. For illustration, during an rTMS session, an electromagnetic coil is placed against a subject's scalp. The electromagnet painlessly delivers a magnetic pulse that stimulates nerve cells in the region of the subject's brain involved in mood control and depression. The rTMS session may also activate regions of the brain that have decreased activity in depression. Empirical evidence suggests that the stimulation can impact how the brain is working, which in turn can ease depression symptoms and improve mood. More contextual information can be obtained from various luminary clinical centers, for example, at mayoclinic.org/tests-procedures/transcranial-magnetic-stimulation/about/pac-20384625.

To measure the impact of TMS on a brain circuit, implementations may interrogate an individual's functional data (such as fMRI data) by analyzing the BOLD signal within two precisely defined three-dimensional regions. In some cases, for a given symptom, one region of the pair corresponds to the region used for TMS targeting and the other region of the pair corresponds to a node of the brain circuit to be modulated by TMS for clinical effect. Here, the therapeutic or clinical effect of TMS is accounted for by a change in the connectivity between the TMS target and the second “downstream” node of a precisely defined circuit. In one example, average BOLD signal across the voxels in each region (TMS target and “downstream node”) can be established and correlated. The value of the correlation between these locations can be compared at any time point to determine if the current TMS parameters are changing circuit connectivity (compared to the value derived for the first, pre-TMS scan). If connectivity is not changing to the extent desired, the parameters for TMS administration can be adapted for modified treatment.

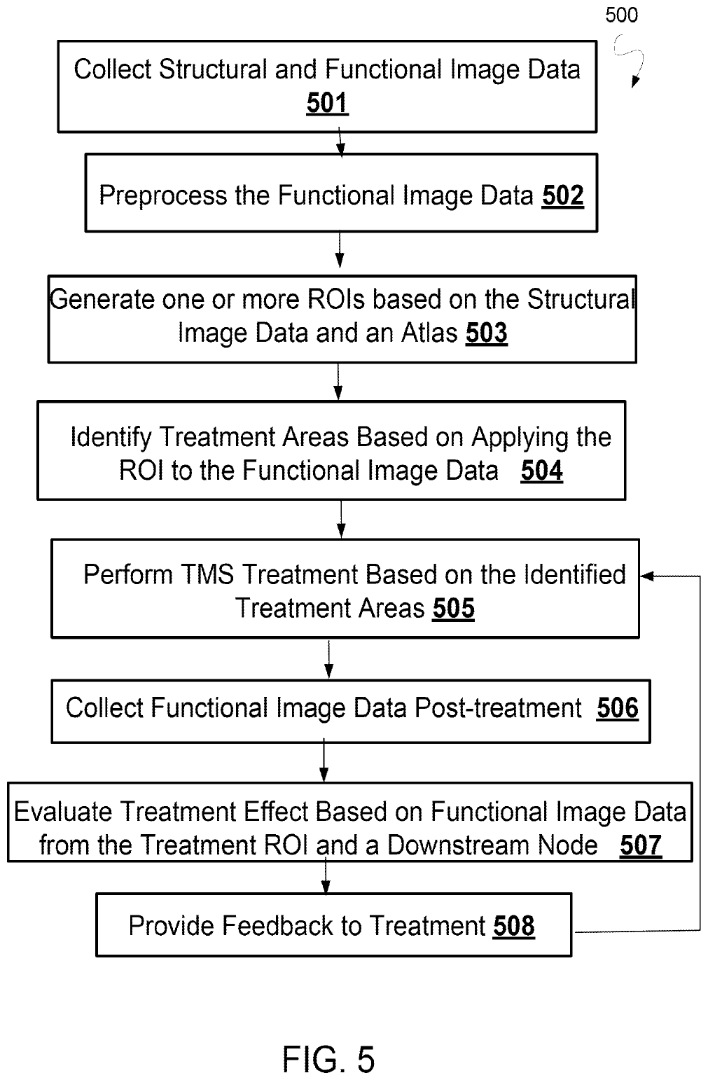

FIG. 5 shows an example of a flow chart 500. Initially, image data may be collected from an individual for the purpose of planning and evaluating transcranial magnetic stimulation (TMS) treatment (501) of a psychotic disorder such as schizophrenia. Schizophrenia is a highly heterogeneous condition and individuals with this disorder vary considerably in their symptoms. These symptoms can be grouped into four different categories: psychotic symptoms (e.g. hallucinations), negative symptoms (asociality, amotivation), cognitive deficits, and mood symptoms.

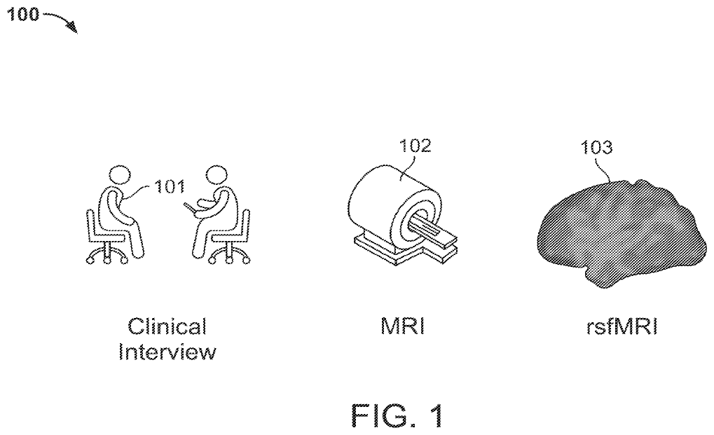

Referring to FIG. 1, diagram 100 shows that a patient can be interviewed by a clinician to evaluate the symptoms (101). The patient may then receive magnetic resonance imaging (MRI) scans (102). The collected images can include two types of images, namely, a structural or static MRI data with a high spatial resolution image, and a resting-state functional MRI (fMRI) data which is a lower spatial resolution set of images. The fMRI data can be acquired by monitoring spontaneous fluctuations in blood oxygen levels in the brain. This process is non-invasive and does not require medication or intravenous dye to collect these images. While prior work using fMRI data has demonstrated that the brain is organized into ˜7 large distributed brain networks that are distinct from each other, the spatial organization of these networks shows substantial variation between individuals. The functional data set (103) can be a 4 dimensional data set that includes multiple images collected over a period of time. In some cases, the 4 dimensional data set can include several (e.g., fewer than 6) minutes of resting-state MRI as well as a structural MRI. The resting state is also known as “task free.” Resting-state fMRI reveals that brain regions within a given network act synchronously with each of each other even when these regions are far apart from each other.

For example, these fMRI image data can be collected on a 3 Tesla MRI scanner any other suitable MRI scanner. In some cases, the high resolution structural image can have a resolution of 1 cubic mm or less. In these cases, the structural MRI image can be a T₁ weighted “structural” MRI image. A “resting-state” or “task-free” functional MRI (fMRI) scan can also be performed. In some cases, the exemplary imaging parameters include: a repetition time (TR) of 3000 milliseconds; an echo time (TE) of 30 milliseconds; a flip angle of 85°; a voxel of 3×3×3-mm “voxels”; and a stack of 47 axial sections in an interleaved acquisition and without a gap. Using these exemplary parameters, sufficient data in an individual can be collected in under 6 minutes of scan time. These parameters serve as an example only. A variety of other imaging sequences can acquire the functional data for planning and evaluation purposes. During the functional scan, the patient may not be required to perform a specific mental task in the scanner. For example, instructions can be given to the patient for the subject to remain still and keep eyes open.

Some implementations preprocess the collected functional data set (502). The pre-processing of, for example, the 4 dimensional data set, can prime the underlying data for analysis. By way of illustration, the preprocessing can isolate blood oxygen level dependent (BOLD) signal that is related to neuronal activity and remove fluctuations in the BOLD signal activity that results from other sources of non-neuronal noise such as, for example, head movement, respiration, heartbeat, or “scanner drift.” For scans with long “repetition time,” which refers to the time it takes to image the entire brain, preprocessing may also include a timing correction to account for the fact that different brain regions are being imaged at different times. These steps can be performed using a variety of software packages.

Other steps in preprocessing can include additional removal of signals that are non-neuronal in origin. Such removal can include removing signals that arise from cerebrospinal fluid and white matter as well as the “global” signal (i.e. the mean signal of the whole brain signal intensity). The resulting data can be temporally filtered to remove fluctuations outside of a defined frequency spectrum, for example, between 0.01 and 0.08 Hz. Finally, the data can be spatially smoothed, for example, by applying a spatial filter. These preprocessing steps can prepare the data for analysis. The exact method of preprocessing is not limited to the above examples and could vary so long as the preprocessing preserves low frequency BOLD signals of neuronal origin.

The next steps (503 and 504) can operate on the collected data that has been preprocessed to generate an individualized target region whose location is specific to the individual patient's anatomy and specific to the symptom being targeted for amelioration. Such location can correspond to, for example, brain circuits that underlie hallucinations, brain circuits that underlie the negative symptoms of schizophrenia, or brain circuits that underlie cognitive deficits.

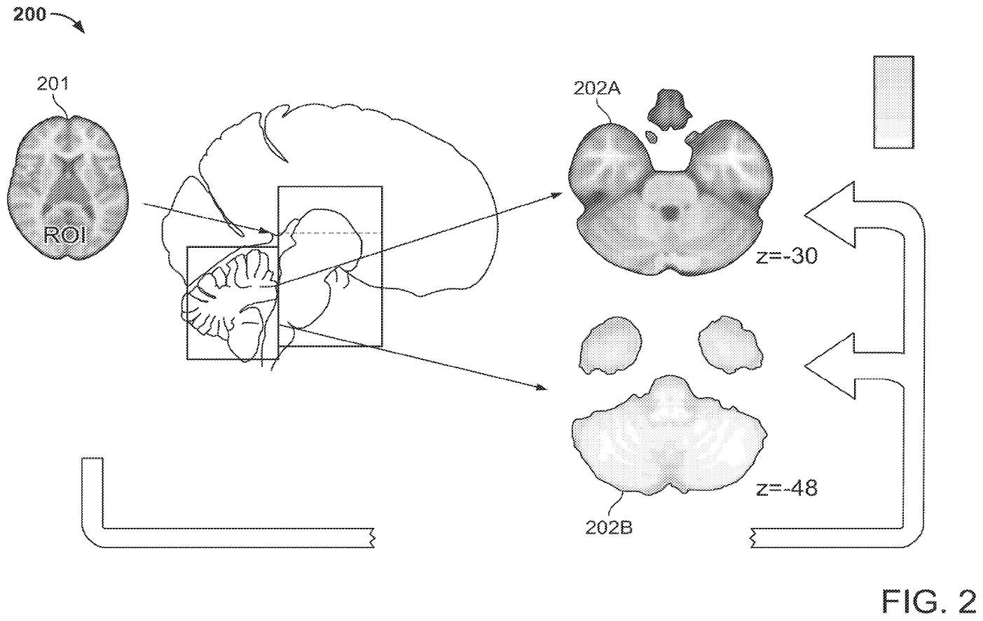

In one example, an individual's fMRI data can be analyzed to determine the BOLD signal within a precisely defined three-dimensional region of interest (ROI), also known as a “mask,” (503). Referring to diagram 200 of FIG. 2, an example of applying the mask is illustrated (201). In some cases, this “mask” ROI can be established based on analyzing relationships between imaging signals and symptom variation in well characterized cohorts of psychotic disorder participants. In other words, this “mask” ROI can based on a pre-determined atlas determined from a cohort of patients. In one example, a recognized symptom region from the atlas can be morphed to the static high-resolution structural image to create a patient-specific and symptom-driven mask. Notably, such masks are not part of a published atlas. In some implementations, these defined 31) mask regions can be specified in a standard coordinate space. For example, these regions are easily visualized and defined in 3D space and in a file format compliant with the Neuroimaging Informatics Technology Initiative (NifTI). In this example, the exact coordinate format can vary depending on the symptom targeted. A non-limiting example can include the coordinates in NifTT format.

Thereafter, the average BOLD signal across the voxels in this 3D mask ROI at each of the time points acquired during the resting state fMRI scan can be correlated with the BOLD signal in every other voxel in the patient's brain images at every time point of the scan (504). Referring to diagram 200 of FIG. 2, an example of performing the correlation between average BOLD signal from voxels in the applied ROI (202A) and the BOLD signal elsewhere in the brain is illustrated (202B). The result is a three-dimensional map of Pearson's correlation coefficients for every voxel in the patient's brain. In some cases, this result is then processed by a threshold to remove spurious readout and allow a visualization of specific structures. In this manner, the spatial distribution of regions of high temporal correlation can be identified. The identification can generate an individual-specific map with sufficient precision to define targets for transcranial magnetic stimulation (TMS). The examples discussed above can three-dimensional maps are symptom specific and distinct and thus allow specific spatial targeting to maximize therapeutic effect for a given symptom. These examples do not limit the specific steps to identify correlated activity. Variations may exist without departing from the innovative spirit for identifying the individual's symptom relevant network to allow for patient and symptom specific TMS targeting.

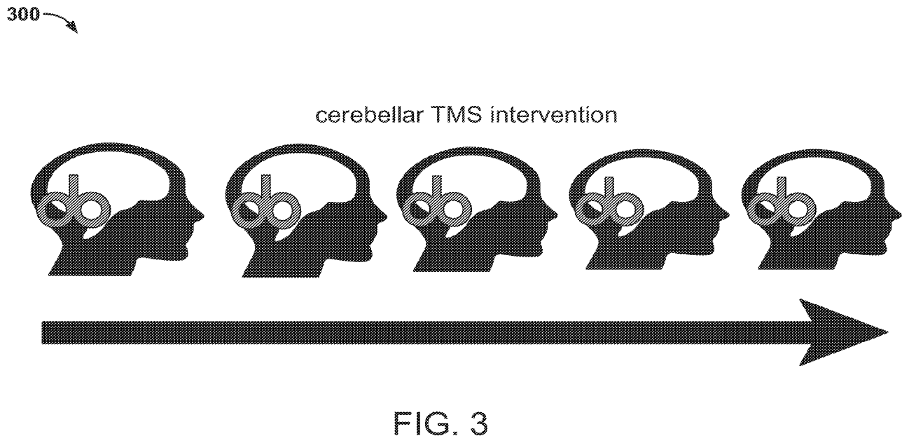

A TMS treatment can be performed based on the identified area that is individually determined and symptom specific (505). Referring to diagram 300 in FIG. 3, an example of using the individually determined and symptom specific target map for applying TMS treatment is illustrated. For example, the three-dimensional image of the patient's brain with the target area can be displayed on a screen during the administration of TMS treatment. In this example, scalp landmarks visualized on the MRI scan can be registered to the position of these landmarks on the patient. A treatment coil can be mounted on the patient's head, who may be held stationary in a treatment room. In the meantime, the on-screen image can be overlaid with the individually identified brain target to allow the TMS treatment to be delivered with the required spatial precision to target the specific area identified for treatment.

In some cases, the individually-determined and symptom-specific target map can provide precise distances from a visible scalp landmark. Because the visible scalp landmark can be located by a TMS operator without the aid of specialized equipment, the scalp landmark thus provides a bridge to use the MRI images as a visual guidance during TMS treatment. For example, the TMS operator can infer the alignment of the treatment area of the TMS coil with the target map on the MM. Additionally or alternatively, implementations can leverage the analysis of the MRI images to manufacture a patient-specific 3D printed template (e.g. a cap or a helmet) to allow precise and reproducible targeting without relying on the provider to manually align with TMS treatment area with the target map. In these implementations, one or more TMS coils can be attached to the 31) printed template, which, once mounted on the patient's head, can allow the TMS treatment area to align with the intended target area as identified from analyzing the MRI images. Non-limiting examples can also include pursuing head-mounted template via software, in that the patient's head and TMS coil can be identified in 3D space with an adaptive system that includes a camera device and a computing device. This adaptive system can use the camera device to obtain images from the subject's head and feed these images to the computing device, for example, in a stream format. The computing device can process the images in view of pre-stored MRI images to infer the alignment and thus correct positioning. Further, the implementations can include targeted application using transcranial ultrasound to treat the target areas, or targeted application using focal electrical stimulation to treat the target areas. For each TMS therapy session, TMS treatment can be performed with a set of stimulation parameters such as, for example, intensity, duration, duty cycle, pulse repetition frequency.

Once targeted TMS treatments have been administered, the outcome of the intervention can be monitored evaluated. While clinical efficacy can be evaluated by patient and the treating physician, the ability to monitor the impact of the TMS treatment on the targeted brain networks allows adjustment and titration of the intervention itself. In other words, if an application of TMS is not impacting the targeted networks as expected, the stimulation parameters of TMS treatment protocol, for example, intensity and frequency/duration, can be adjusted accordingly to optimize the outcome of a TMS therapy session.

In some implementations, the impact of TMS treatment on the targeted brain network area can be measured. In these implementations, the participant may be assumed to have had pre-treatment fMRI imaging as described above for the purposes of TMS target determination. At a time after the TMS treatment when, for example, a clinicians would like to assess the impact of TMS on symptom specific brain circuits, the participant may undergo a fMRI imaging session when the participant is in a resting state (506). This fMRI imaging session can be a repeat of the resting-state MRI as explained above. Similar to the earlier described imaging session, the exact details of the imaging sequence may vary so long as this “follow-up” imaging sequence uses the same imaging parameters as the original “targeting” IRI scan. In other words, the follow-up imaging sequence can use a protocol that is identical to the resting-state MRI as explained above. This follow-up imaging may likewise be preprocessed in the same manner as the initial fMRI targeting scan and, as before, exact parameters may vary as long as the same protocol is used to preprocess the individual's fMRI data before and after TMS treatment.

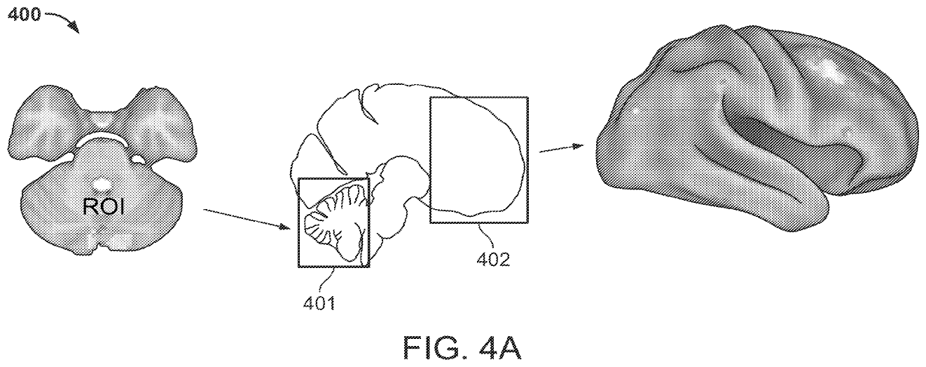

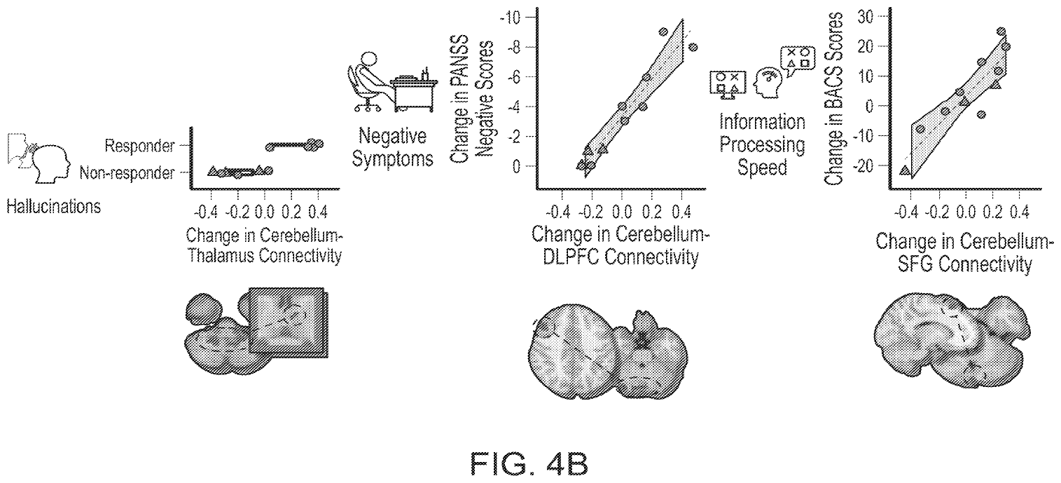

Referring to diagram 400 of FIG. 4A, an example of analyzing a follow-up fMRI scan is provided. As illustrated, the BOLD signals within two precisely defined three-dimensional regions, namely, region 401 and region 402, of the patient's brain are analyzed. Regions 401 and 402 may vary according to the specific symptom targeted. For a given symptom, one region of the pair corresponds to the region used for TMS targeting, which has been identified in step 504 as explained above. The other region of the pair is a node of the brain circuit to be modulated by the TMS treatment for clinical effect. In many cases, the therapeutic or clinical effect of the TMS treatment can be evaluated by a change in the connectivity between the TMS target and the second “downstream” node of a precisely defined circuit. Similar to the TMS targets, which are symptom specific, the corresponding “downstream nodes” are also symptom specific. These regions (or ROIs) are likewise known as masks, none of which has been part of a published atlas. In many cases, these defined 3D regions are specified in a standard coordinate space for storage and retrieval purposes. For example, these regions can be visualized and defined in a NifTI file. Other formats may also be used so long as a 3D space is specified. The exact coordinates specified vary depending on the symptom targeted.

For an individual, the average BOLD signal across the voxels in each region can be correlated at each time point during the fMRI scan. For example, region 401 used for TMS target and region 402 known as the downstream node, as acquired during the resting state fMRI scan, can be preprocessed and analyzed to extract the average BOLD signal. Once completed, some implementations may perform a correlation between the average BOLD signal strength in each region across all time points during the fMRI scan. This calculation is performed on the fMRI imaging data collected at baseline and at all subsequent fMRI sessions acquired in the course of treatment, which can take multiple sessions of TMS treatment. The value of the correlation between these locations can be compared at any time point to determine if the current THIS parameters are changing circuit connectivity, as compared to, for example, the value derived for the first, pre-TMS scan. In this manner, the effect of the TMS treatment can be evaluated based on the functional image data from the treatment ROI and a downstream node (507). This evaluation can provide feedback to the treating physician (508) such that subsequent TMS treatment may be administered with altered parameters. For example, if connectivity is not changing to the extent desired, the treating physician may evaluate the data and make changes to the parameters of subsequent TMS administration as indicated. Indeed, implementations described in the present disclosure may optimize or fine tune repetitive TMS (rTMS) for more expeditious treatment outcome.

FIG. 4B shows our preliminary results comparing observed changes in symptoms with changes in circuit targets after (EMS therapy. Each rounded dot represents a subject from the treatment group while each triangle represents a subject from the sham group. In the left panel, TMS induced increase in circuit target connectivity (x-axis) was linked to a reduction in hallucination symptoms and active TMS was more effective than sham TMS in improving connectivity in this circuit target. Regarding negative symptoms of schizophrenia (e.g., amotivation and anhedonia), the middle panel shows a distinct circuit target is linked to negative symptom severity as measured by the Positive and Negative Syndrome Scale (PANSS). Reduction in symptom severity (vertical axis) was linked to TMS induced increase in connectivity in a distinct circuit target (horizontal axis) and the active TMS group demonstrated a greater increase in target circuit connectivity than the sham group. In the right panel, a distinct target circuit is linked to cognitive symptoms in psychotic disorders. Here, change in information processing speed (measured by BACS symbol coding task, vertical axis) is linked to change in connectivity in a circuit target distinct from the other circuit targets. At a later (three-week follow-up) time point the active TMS group demonstrated a faster processing speed than the sham TMS group.

For context, psychotic disorders such as schizophrenia are chronic, disabling psychiatric disorders characterized by psychosis, ‘negative symptoms’ (e.g. amotivation), cognitive deficits, and mood symptoms. A combination of these symptoms is a defining characteristic of schizophrenia, but individuals are very heterogenous in specific symptoms and severity.

The standard of care for treating schizophrenia is the prescription of antipsychotic medications. This has been the standard of care for 65 years without any new development of new classes of medications or substantial improvements during that time. These drugs only treat a subset of schizophrenia symptoms (i.e. the psychotic symptoms) and leave the most disabling symptoms (i.e. negative symptoms like asociality and amotivation plus cognitive deficits) completely untouched. These medications carry a heavy side-effect burden and as a result, the average patient finds any antipsychotic both efficacious and tolerable only ˜30% of the time. This number has essentially unchanged over a half century of drug development. The apparent lack of progress despite 50±years of development is largely caused by the fact that the calming effects of these known drugs and their development was not derived from any understanding of the biology of schizophrenia. There is little reason to think that their mechanism of action is actually correcting an underlying deficit in this disease.

The neural circuit basis (e.g., cerebellar-cortical circuit dysfunction) for these phenotypes render it conceivable to localize and manipulate the underlying neural circuits in participants who have reduced processing speed, or are diagnosed with a psychotic disorder or at high risk for developing a psychotic disorder. Various implementations described in the present disclosure may rescue the circuit connectivity by neuromodulation on an individual basis and specific to a symptom. Indeed, implementations described by the present disclosure provide a fundamentally completely different approach to treating psychotic disorders by attempting to actually correct the underlying pathology of this disease. This shifted mechanism can lead to a reduction in psychotic symptoms as well as so called negative symptoms and cognitive deficits. As such, the implementations described by the present disclosure outline a treatment for symptoms for which there is no current standard of care (i.e. these symptoms have no current medication treatments). For example, implementations described in the present disclosure involves the individualized identification of specific brain networks in a patient with schizophrenia and then restoring the function of these brain networks through the use of non-invasive brain stimulation. In other words, implementations described in the present disclosure provide a focused and non-invasive intervention that targets the brain circuit pathology of this disease, capable of avoiding the intolerable and systemic side effects that accompany conventional antipsychotic medications.

In more detail, each of the different symptoms of schizophrenia (psychotic, negative, cognitive, mood) are the result of dysfunctional brain circuits, which means that these circuits are symptom specific and distinct i.e. a breakdown in a given circuit can cause one symptom but not the others. Implementations described in the present disclosure are capable of identifying, targeting and modifying the brain networks that give rise to the symptoms of schizophrenia so that these symptoms may be ameliorated in persons diagnosed with this disorder. The precise identification of these networks is significant for several reasons. First, deficits in a specific brain network can give rise to specific symptoms. Second, individuals vary significantly from each other in the spatial organization (or location) of these brain networks. Third, individuals vary significantly in their specific symptoms related to schizophrenia and therefore the network(s) of interest can vary between patients depending on their individual clinical needs. Fourth, the TMS effects are focal and there is evidence that small differences in TMS targeting lead to significant differences in clinical effects.

Various implementations can identify these circuits in patients with psychotic disorders on an individual basis and specific to each symptom. This identification can be precise enough to allow targeted intervention to restore function in these circuits using a non-invasive technique called transcranial magnetic stimulation (TMS). By targeting TMS treatment to the identified circuit, the implementations can restore function in these circuits, thereby reducing specific symptoms. This effect of such targeted treatment may not be confined to a single symptom domain. As such, the implementations can reduce the medication-refractory ‘negative’ symptoms and cognitive deficits of schizophrenia in addition to psychotic symptoms.

Implementations can use a rapidly alternating magnetic field to generate electrical activity in the brain in a spatially specific region. While TMS has already been used therapeutically for depression, TMS treatment for depression has not been known to use brain data for targeting purposes. For this reason, known TMS treatment protocols may not allow individual-specific targeting of specific brain circuits. In comparison, various implementations of the present disclosure allow individual-specific maps of networks in the cerebellum to be visualized and used to guide placement of the TMS coil to modulate the specific brain circuit targeted and therefore reduce the schizophrenia symptoms targeted for intervention. Such targeted application of TMS may even reverse symptoms of schizophrenia. The effect of the TMS treatment can be evaluated by a follow-up imaging of the patient's brain and subsequent image analysis to determine circuit restoration. The analysis can provide feedback to additional TMS treatment. In this manner, a tailored rTMS protocol can be established based on feedback. For example, the details of the duration of TMS treatment and the number or frequency of TMS sessions may vary based on the feedback. Moreover, such treatment protocol may vary from patient to patient based on individual symptoms and goals of treatment.

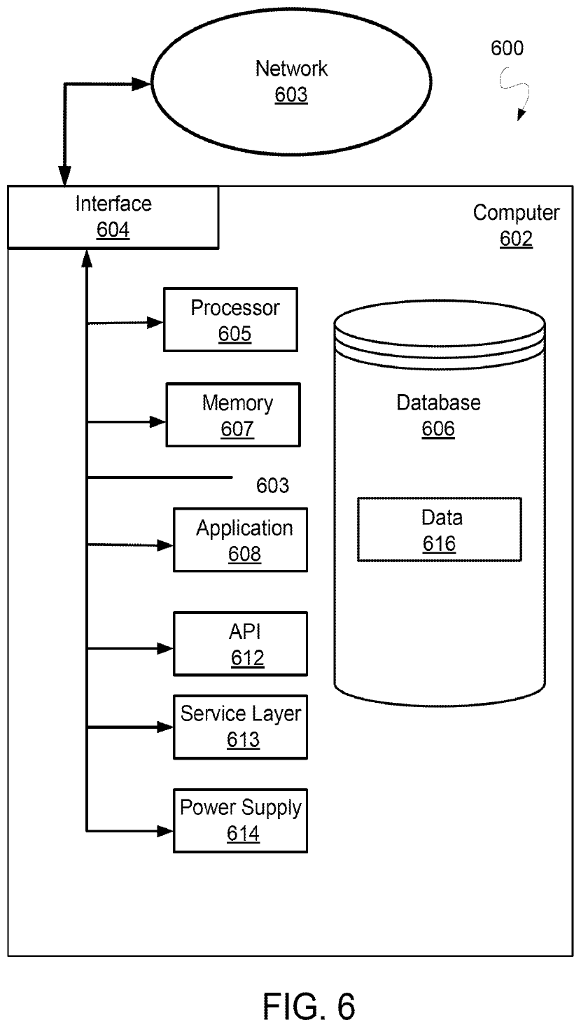

FIG. 6 is a block diagram illustrating an example of a computer system 600 used to provide computational functionalities associated with described algorithms, methods, functions, processes, flows, and procedures, according to an implementation of the present disclosure. The illustrated computer 602 is intended to encompass any computing device such as a server, desktop computer, laptop/notebook computer, wireless data port, smart phone, personal data assistant (PDA), tablet computing device, one or more processors within these devices, another computing device, or a combination of computing devices, including physical or virtual instances of the computing device, or a combination of physical or virtual instances of the computing device. Additionally, the computer 602 can comprise a computer that includes an input device, such as a keypad, keyboard, touch screen, another input device, or a combination of input devices that can accept user information, and an output device that conveys information associated with the operation of the computer 602, including digital data, visual, audio, another type of information, or a combination of types of information, on a graphical-type user interface (UI) (or GUI) or other UI.

The computer 602 can serve in a role in a computer system as a client, network component, a server, a database or another persistency, another role, or a combination of roles for performing the subject matter described in the present disclosure. The illustrated computer 602 is communicably coupled with a network 603. In some implementations, one or more components of the computer 602 can be configured to operate within an environment, including cloud-computing-based, local, global, another environment, or a combination of environments.

The computer 602 is an electronic computing device operable to receive, transmit, process, store, or manage data and information associated with the described subject matter. According to some implementations, the computer 602 can also include or be communicably coupled with a server, including an application server, e-mail server, web server, caching server, streaming data server, another server, or a combination of servers.

The computer 602 can receive requests over network 603 (for example, from a client software application executing on another computer 602) and respond to the received requests by processing the received requests using a software application or a combination of software applications. In addition, requests can also be sent to the computer 602 from internal users, external or third-parties, or other entities, individuals, systems, or computers.

Each of the components of the computer 602 can communicate using a system bus 603. In some implementations, any or all of the components of the computer 602, including hardware, software, or a combination of hardware and software, can interface over the system bus 603 using an application programming interface (API) 612, a service layer 613, or a combination of the API 612 and service layer 613. The API 612 can include specifications for routines, data structures, and object classes. The API 612 can be either computer-language independent or dependent and refer to a complete interface, a single function, or even a set of APIs. The service layer 613 provides software services to the computer 602 or other components (whether illustrated or not) that are communicably coupled to the computer 602. The functionality of the computer 602 can be accessible for all service consumers using this service layer. Software services, such as those provided by the service layer 613, provide reusable, defined functionalities through a defined interface. For example, the interface can be software written in JAVA, C++, another computing language, or a combination of computing languages providing data in extensible markup language (XML) format, another format, or a combination of formats. While illustrated as an integrated component of the computer 602, alternative implementations can illustrate the API 612 or the service layer 613 as stand-alone components in relation to other components of the computer 602 or other components (whether illustrated or not) that are communicably coupled to the computer 602. Moreover, any or all parts of the API 612 or the service layer 613 can be implemented as a child or a sub-module of another software module, enterprise application, or hardware module without departing from the scope of the present disclosure.

The computer 602 includes an interface 604. Although illustrated as a single interface 604 in FIG. 6, two or more interfaces 604 can be used according to particular needs, desires, or particular implementations of the computer 602. The interface 604 is used by the computer 602 for communicating with another computing system (whether illustrated or not) that is communicatively linked to the network 603 in a distributed environment. Generally, the interface 604 is operable to communicate with the network 603 and comprises logic encoded in software, hardware, or a combination of software and hardware. More specifically, the interface 604 can comprise software supporting one or more communication protocols associated with communications such that the network 603 or interface's hardware is operable to communicate physical signals within and outside of the illustrated computer 602.

The computer 602 includes a processor 605. Although illustrated as a single processor 605 in FIG. 6, two or more processors can be used according to particular needs, desires, or particular implementations of the computer 602. Generally, the processor 605 executes instructions and manipulates data to perform the operations of the computer 602 and any algorithms, methods, functions, processes, flows, and procedures as described in the present disclosure.

The computer 602 also includes a database 606 that can hold data for the computer 602, another component communicatively linked to the network 603 (whether illustrated or not), or a combination of the computer 602 and another component. For example, database 606 can be an in-memory, conventional, or another type of database storing data consistent with the present disclosure. In some implementations, database 606 can be a combination of two or more different database types (for example, a hybrid in-memory and conventional database) according to particular needs, desires, or particular implementations of the computer 602 and the described functionality. Although illustrated as a single database 606 in FIG. 6, two or more databases of similar or differing types can be used according to particular needs, desires, or particular implementations of the computer 602 and the described functionality. While database 606 is illustrated as an integral component of the computer 602, in alternative implementations, database 606 can be external to the computer 602. As illustrated, the database 606 holds data 616, which, as described previously, can include, for example, the high resolution structural image, the 4 dimensional fMRI images based on which the treatment ROI is identified, the 4 dimensional fMRI images based on which post-treatment evaluation is performed, the atlas of a mask ROI derived from a cohort of patients in order to derive the treatment ROI, and the atlas of various downstream nodes for follow-up analysis to evaluate the effect of TMS treatment.

The computer 602 also includes a memory 607 that can hold data for the computer 602, another component or components communicatively linked to the network 603 (whether illustrated or not), or a combination of the computer 602 and another component. Memory 607 can store any data consistent with the present disclosure. In some implementations, memory 607 can be a combination of two or more different types of memory (for example, a combination of semiconductor and magnetic storage) according to particular needs, desires, or particular implementations of the computer 602 and the described functionality. Although illustrated as a single memory 607 in FIG. 6, two or more memories 607 or similar or differing types can be used according to particular needs, desires, or particular implementations of the computer 602 and the described functionality. While memory 607 is illustrated as an integral component of the computer 602, in alternative implementations, memory 607 can be external to the computer 602.

The application 608 is an algorithmic software engine providing functionality according to particular needs, desires, or particular implementations of the computer 602, particularly with respect to functionality described in the present disclosure. For example, application 608 can serve as one or more components, modules, or applications. Further, although illustrated as a single application 608, the application 608 can be implemented as multiple applications 608 on the computer 602. In addition, although illustrated as integral to the computer 602, in alternative implementations, the application 608 can be external to the computer 602.

The computer 602 can also include a power supply 614. The power supply 614 can include a rechargeable or non-rechargeable battery that can be configured to be either user- or non-user-replaceable. In some implementations, the power supply 614 can include power-conversion or management circuits (including recharging, standby, or another power management functionality). In some implementations, the power-supply 614 can include a power plug to allow the computer 602 to be plugged into a wall socket or another power source to, for example, power the computer 602 or recharge a rechargeable battery.

There can be any number of computers 602 associated with, or external to, a computer system containing computer 602, each computer 602 communicating over network 603. Further, the term “client,” “user,” or other appropriate terminology can be used interchangeably, as appropriate, without departing from the scope of the present disclosure. Moreover, the present disclosure contemplates that many users can use one computer 602, or that one user can use multiple computers 602.

Implementations of the subject matter and the functional operations described in this specification can be implemented in digital electronic circuitry, in tangibly embodied computer software or firmware, in computer hardware, including the structures disclosed in this specification and their structural equivalents, or in combinations of one or more of them. Software implementations of the described subject matter can be implemented as one or more computer programs, that is, one or more modules of computer program instructions encoded on a tangible, non-transitory, computer-readable computer-storage medium for execution by, or to control the operation of, data processing apparatus. Alternatively, or additionally, the program instructions can be encoded in/on an artificially generated propagated signal, for example, a machine-generated electrical, optical, or electromagnetic signal that is generated to encode information for transmission to a receiver apparatus for execution by a data processing apparatus. The computer-storage medium can be a machine-readable storage device, a machine-readable storage substrate, a random or serial access memory device, or a combination of computer-storage mediums. Configuring one or more computers means that the one or more computers have installed hardware, firmware, or software (or combinations of hardware, firmware, and software) so that when the software is executed by the one or more computers, particular computing operations are performed.

The term “real-time,” “real time,” “realtime,” “real (fast) time (RFT),” “near(ly) real-time (NRT),” “quasi real-time,” or similar terms (as understood by one of ordinary skill in the art), means that an action and a response are temporally proximate such that an individual perceives the action and the response occurring substantially simultaneously. For example, the time difference for a response to display (or for an initiation of a display) of data following the individual's action to access the data can be less than 1 millisecond (ms), less than 1 second (s), or less than 5 s. While the requested data need not be displayed (or initiated for display) instantaneously, it is displayed (or initiated for display) without any intentional delay, taking into account processing limitations of a described computing system and time required to, for example, gather, accurately measure, analyze, process, store, or transmit the data.

The terms “data processing apparatus,” “computer,” or “electronic computer device” (or equivalent as understood by one of ordinary skill in the art) refer to data processing hardware and encompass all kinds of apparatus, devices, and machines for processing data, including by way of example, a programmable processor, a computer, or multiple processors or computers. The apparatus can also be, or further include special purpose logic circuitry, for example, a central processing unit (CPU), an FPGA (field programmable gate array), or an ASIC (application-specific integrated circuit). In some implementations, the data processing apparatus or special purpose logic circuitry (or a combination of the data processing apparatus or special purpose logic circuitry) can be hardware- or software-based (or a combination of both hardware- and software-based). The apparatus can optionally include code that creates an execution environment for computer programs, for example, code that constitutes processor firmware, a protocol stack, a database management system, an operating system, or a combination of execution environments. The present disclosure contemplates the use of data processing apparatuses with an operating system of some type, for example LINUX, UNIX, WINDOWS, MAC OS, ANDROID, IOS, another operating system, or a combination of operating systems.

A computer program, which can also be referred to or described as a program, software, a software application, a unit, a module, a software module, a script, code, or other component can be written in any form of programming language, including compiled or interpreted languages, or declarative or procedural languages, and it can be deployed in any form, including, for example, as a stand-alone program, module, component, or subroutine, for use in a computing environment. A computer program can, but need not, correspond to a file in a file system. A program can be stored in a portion of a file that holds other programs or data, for example, one or more scripts stored in a markup language document, in a single file dedicated to the program in question, or in multiple coordinated files, for example, files that store one or more modules, sub-programs, or portions of code. A computer program can be deployed to be executed on one computer or on multiple computers that are located at one site or distributed across multiple sites and interconnected by a communication network.

While portions of the programs illustrated in the various figures can be illustrated as individual components, such as units or modules, that implement described features and functionality using various objects, methods, or other processes, the programs can instead include a number of sub-units, sub-modules, third-party services, components, libraries, and other components, as appropriate. Conversely, the features and functionality of various components can be combined into single components, as appropriate. Thresholds used to make computational determinations can be statically, dynamically, or both statically and dynamically determined.

Described methods, processes, or logic flows represent one or more examples of functionality consistent with the present disclosure and are not intended to limit the disclosure to the described or illustrated implementations, but to be accorded the widest scope consistent with described principles and features. The described methods, processes, or logic flows can be performed by one or more programmable computers executing one or more computer programs to perform functions by operating on input data and generating output data. The methods, processes, or logic flows can also be performed by, and apparatus can also be implemented as, special purpose logic circuitry, for example, a CPU, an FPGA, or an ASIC.

Computers for the execution of a computer program can be based on general or special purpose microprocessors, both, or another type of CPU. Generally, a CPU will receive instructions and data from and write to a memory. The essential elements of a computer are a CPU, for performing or executing instructions, and one or more memory devices for storing instructions and data. Generally, a computer will also include, or be operatively coupled to, receive data from or transfer data to, or both, one or more mass storage devices for storing data, for example, magnetic, magneto-optical disks, or optical disks. However, a computer need not have such devices. Moreover, a computer can be embedded in another device, for example, a mobile telephone, a personal digital assistant (PDA), a mobile audio or video player, a game console, a global positioning system (GPS) receiver, or a portable memory storage device.

Non-transitory computer-readable media for storing computer program instructions and data can include all forms of media and memory devices, magnetic devices, magneto optical disks, and optical memory device. Memory devices include semiconductor memory devices, for example, random access memory (RAM), read-only memory (ROM), phase change memory (PRAM), static random access memory (SRAM), dynamic random access memory (DRAM), erasable programmable read-only memory (EPROM), electrically erasable programmable read-only memory (EEPROM), and flash memory devices. Magnetic devices include, for example, tape, cartridges, cassettes, internal/removable disks. Optical memory devices include, for example, digital video disc (DVD), CD-ROM, DVD+/−R, DVD-RAM, DVD-ROM, HD-DVD, and BLURAY, and other optical memory technologies. The memory can store various objects or data, including caches, classes, frameworks, applications, modules, backup data, jobs, web pages, web page templates, data structures, database tables, repositories storing dynamic information, or other appropriate information including any parameters, variables, algorithms, instructions, rules, constraints, or references. Additionally, the memory can include other appropriate data, such as logs, policies, security or access data, or reporting files. The processor and the memory can be supplemented by, or incorporated in, special purpose logic circuitry.

To provide for interaction with a user, implementations of the subject matter described in this specification can be implemented on a computer having a display device, for example, a CRT (cathode ray tube), LCD (liquid crystal display), LED (Light Emitting Diode), or plasma monitor, for displaying information to the user and a keyboard and a pointing device, for example, a mouse, trackball, or trackpad by which the user can provide input to the computer. Input can also be provided to the computer using a touchscreen, such as a tablet computer surface with pressure sensitivity, a multi-touch screen using capacitive or electric sensing, or another type of touchscreen. Other types of devices can be used to interact with the user. For example, feedback provided to the user can be any form of sensory feedback. Input from the user can be received in any form, including acoustic, speech, or tactile input. In addition, a computer can interact with the user by sending documents to and receiving documents from a client computing device that is used by the user.

The term “graphical user interface,” or “GUI,” can be used in the singular or the plural to describe one or more graphical user interfaces and each of the displays of a particular graphical user interface. Therefore, a GUI can represent any graphical user interface, including but not limited to, a web browser, a touch screen, or a command line interface (CLI) that processes information and efficiently presents the information results to the user. In general, a GUI can include a plurality of user interface (UI) elements, some or all associated with a web browser, such as interactive fields, pull-down lists, and buttons. These and other UI elements can be related to or represent the functions of the web browser.

Implementations of the subject matter described in this specification can be implemented in a computing system that includes a back-end component, for example, as a data server, or that includes a middleware component, for example, an application server, or that includes a front-end component, for example, a client computer having a graphical user interface or a Web browser through which a user can interact with an implementation of the subject matter described in this specification, or any combination of one or more such back-end, middleware, or front-end components. The components of the system can be interconnected by any form or medium of wireline or wireless digital data communication (or a combination of data communication), for example, a communication network. Examples of communication networks include a local area network (LAN), a radio access network (RAN), a metropolitan area network (MAN), a wide area network (WAN), Worldwide Interoperability for Microwave Access (WIMAX), a wireless local area network (WLAN) using, for example, 802.11 a/b/g/n or 802.20 (or a combination of 802.11x and 802.20 or other protocols consistent with the present disclosure), all or a portion of the Internet, another communication network, or a combination of communication networks. The communication network can communicate with, for example, Internet Protocol (IP) packets, Frame Relay frames, Asynchronous Transfer Mode (ATM) cells, voice, video, data, or other information between networks addresses.

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of what can be claimed, but rather as descriptions of features that can be specific to particular implementations. Certain features that are described in this specification in the context of separate implementations can also be implemented, in combination, in a single implementation. Conversely, various features that are described in the context of a single implementation can also be implemented in multiple implementations, separately, or in any sub-combination. Moreover, although previously described features can be described as acting in certain combinations and even initially claimed as such, one or more features from a claimed combination can, in some cases, be excised from the combination, and the claimed combination can be directed to a sub-combination or variation of a sub-combination.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations can be considered optional), to achieve desirable results. In certain circumstances, multitasking or parallel processing (or a combination of multitasking and parallel processing) can be advantageous and performed as deemed appropriate.

Moreover, the separation or integration of various system modules and components in the previously described implementations should not be understood as requiring such separation or integration in all implementations, and it should be understood that the described program components and systems can generally be integrated together in a single software product or packaged into multiple software products.

Furthermore, any claimed implementation is considered to be applicable to at least a computer-implemented method; a non-transitory, computer-readable medium storing computer-readable instructions to perform the computer-implemented method; and a computer system comprising a computer memory interoperably coupled with a hardware processor configured to perform the computer-implemented method or the instructions stored on the non-transitory, computer-readable medium.

What is claimed is:

1. A computer-implemented method comprising:
accessing brain images of a patient who has been diagnosed with a psychotic disorder, or who is at high risk for developing the psychotic disorder, wherein the brain images comprise a first set of images depicting a brain structure of the patient and a second set of images encoding a brain function of the patient;
generating one or more masks based on morphing a symptom region of an atlas to the first set of images, wherein the symptom region of the atlas is identified from a cohort of participants with the psychotic disorder;
identifying one or more treatment regions based on applying the one or more masks to the second set of images, wherein the treatment regions are specific to the patient and a symptom of the psychotic disorder; and
causing at least one transcranial magnetic stimulation (TMS) therapy session targeting the one or more treatment regions to be administered to a brain of the patient.

2. The method of claim 1, further comprising:
accessing a third set of images of the patient that encode the brain function of the patient after the at least one TMS therapy session is administered.

3. The method of claim 2, further comprising:
applying the one or more treatment regions to the third set of images; and
evaluating an effect of the at least one TMS therapy session based on results of correlating a signal from the one or more treatment regions and a signal from at least one node that is downstream of the one or more treatment regions.

4. The method of claim 3, further comprising:
causing at least one more TMS therapy session targeting the one or more treatment regions to be administered to the patient, wherein at least one stimulation parameter of the at least one more TMS therapy session is updated based on results of the evaluation.

5. The method of claim 2, wherein the first set of images are characterized by a spatial resolution higher than the second set of images.

6. The method of claim 5, wherein the second set of images comprise functional magnetic resonance imaging (fMRI) images that encode the brain function of the patient.

7. The method of claim 6, further comprising:
removing signals of non-neuronal origin from the fMRI images based on pre-processing.

8. The method of claim 6, wherein identifying one or more treatment regions comprises:
performing a correlation between an average blood oxygen level dependent (BOLD) signal from voxels within the one or more masks applied to the second set of images and a BOLD signal elsewhere in the second set of images.

9. The method of claim 8, wherein identifying one or more treatment regions further comprises:
applying a threshold to results of the correlation such that the one or more treatment regions specific to the patient and the symptom of the psychotic disorder can be identified.

10. The method of claim 6, wherein the third set of images comprise fMRI images encoding the brain function of the patient, wherein the third set of images and the second set of images are acquired using an identical protocol.

11. The method of claim 10, further comprising:
performing a correlation between an average BOLD signal from voxels within the one or more masks applied to the third set of images and a BOLD signal from voxels within a downstream node within the third set of images; and
evaluating an effect of the at least one TMS therapy session based on results of the correlation.

12. The method of claim 1, further comprising:
guiding the at least one TMS therapy session based on the one or more treatment regions.

13. The method of claim 12, further comprising:
generating a head-mounted template which, when mounted on the patient along with one or more TMS coil, causes the one or more TMS coil to focus on the one or more treatment regions within the brain of the patient.

14. A computer system comprising one or more processors configured to perform operations of:
accessing brain images of a patient who has been diagnosed with a psychotic disorder, or who is at high risk for developing the psychotic disorder, wherein the brain images comprise a first set of images depicting a brain structure of the patient and a second set of images encoding a brain function of the patient;
generating one or more masks based on morphing a symptom region of an atlas to the first set of images, wherein the symptom region of the atlas is identified from a cohort of participants with the psychotic disorder;
identifying one or more treatment regions based on applying the one or more masks to the second set of images, wherein the treatment regions are specific to the patient and a symptom of the psychotic disorder; and
causing at least one transcranial magnetic stimulation (TMS) therapy session targeting the one or more treatment regions to be administered to a brain of the patient.

15. The computer system of claim 14, wherein the operations further comprise:
accessing a third set of images of the patient that encode the brain function of the patient after the at least one TMS therapy session is administered.

16. The computer system of claim 15, wherein the operations further comprise:
applying the one or more treatment regions to the third set of images; and
evaluating an effect of the at least one TMS therapy session based on results of correlating a signal from the one or more treatment regions and a signal from at least one node that is downstream of the one or more treatment regions.

17. The computer system of claim 16, wherein the operations further comprise:
causing at least one more TMS therapy session targeting the one or more treatment regions to be administered to the patient, wherein at least one stimulation parameter of the at least one more TMS therapy session is updated based on results of the evaluation.

18. The computer system of claim 15, wherein the first set of images are characterized by a spatial resolution higher than the second set of images.

19. The computer system of claim 18, wherein the second set of images comprise functional magnetic resonance imaging (fMRI) images that encode the brain function of the patient.

20. The computer system of claim 19, wherein the operations further comprise:
removing signals of non-neuronal origin from the fMRI images based on pre-processing.

21. The computer system of claim 19, wherein the operations further comprise:
performing a correlation between an average blood oxygen level dependent (BOLD) signal from voxels within the one or more masks applied to the second set of images and a BOLD signal elsewhere in the second set of images.

22. The computer system of claim 21, wherein the operations further comprise:
applying a threshold to results of the correlation such that the one or more treatment regions specific to the patient and the psychotic disorder can be identified.

23. The computer system of claim 19, wherein the third set of images comprise fMRI images encoding the brain function of the patient, wherein the third set of images and the second set of images are acquired using an identical protocol.

24. The computer system of claim 23, wherein the operations further comprise:
performing a correlation between an average BOLD signal from voxels within the one or more masks applied to the third set of images and a BOLD signal from voxels within a downstream node within the third set of images; and
evaluating an effect of the at least one TMS therapy session based on results of the correlation.

25. The computer system of claim 14, wherein the operations further comprise:
guiding the at least one TMS therapy session based on the one or more treatment regions.

26. The computer system of claim 25, wherein the operations further comprise:
generating a head-mounted template which, when mounted on the patient along with one or more TMS coil, causes the one or more TMS coil to focus on the one or more treatment regions within the brain of the patient.
